(19) United States
(12) Patent Application Publication
(10) Pub. No.: US 2019/0046369 A1
(43) Pub. Date: Feb. 14, 2019

(54) INCONTINENCE PRODUCT

(71) Applicant: KUVER DESIGNS PTY LTD, Sandy Bay, Tasmania (AU)

(72) Inventor: Susan M. Allison-Rogers, Tasmania (AU)

(73) Assignee: KUVER DESIGNS PTY LTD, Sandy Bay, Tasmania (AU)

(21) Appl. No.: 16/080,272

(22) PCT Filed: Mar. 7, 2017
(86) PCT No.: PCT/AU2017/050196

§ 371 Date: Aug. 27, 2018

(30) Foreign Application Priority Data
Mar. 7, 2016 (AU) 2016900833

(51) Int. Cl. A61F 13/74; A61F 13/49; A61F 13/62

(52) U.S. Cl. CPC A61F 13/74; A61F 13/49004; A61F 13/565; A61F 13/49006; A61F 13/622

(57) ABSTRACT

An incontinence product for a baby or a child or an adult. The product comprises a disposable pad that is shaped to fit around a crotch region of a person wearing the product. The product also comprises a reusable out pull-on pant having hook fasteners on the inside body of the pant to engage an outer cover stock of the disposable pad and thereby retain the pad in an operative position.

### TECHNICAL FIELD

The present invention relates to an incontinence product for a baby or a child or an adult.

### BACKGROUND ART

The market for incontinence products for babies, children and adults is a substantial market and there are significant practical, personal and environmental (i.e. product disposal) issues associated with the current products that are supplied to the market.

There is a need for an incontinence product that is easier to manufacture, easier to use and more environmentally sustainable than the current products.

### SUMMARY OF THE INVENTION

In broad terms the present invention provides an incontinence product for a baby or a child or an adult that comprises:

(a) a disposable pad that is shaped to fit around a crotch region of a person wearing the product; and

(b) a reusable outer pull-on pant having hook fasteners on an inside surface of a body of the pant to engage an outer cover stock of the disposable pad and thereby retain the pad in an operative position.

The term “reusable” is understood herein to mean that the pull-on pant can be used more than once and then either discarded (disposable) or washed and reused.

The fitting sequence for a new pad as a replacement for an existing pad of the above product on the person is simple and straightforward. It is possible to replace pads quickly and easily, with minimal disturbance to clothing. In particular, it is not necessary for the person to remove trousers or stockings to replace the pad. The person can simply pull down trousers or stockings, pull down the pull-on pant, remove an existing pad, position a new pad, pull up the pull-on pant and re-arrange the pad as required to ensure the pad is located properly, and pull-up trousers or stockings.

In addition, the overall product design and construction is simple and effective. The pull-on pant is suited particularly well to retaining the disposable pad in position on the person.

The use of the hook fasteners on the pull-on pant rather than having fasteners on the pad makes the construction of the pad straightforward. In particular, it is not necessary to complicate the structure (and manufacturing cost) of the pad by providing the pad with specific fastening systems. This is a significant difference between the product of the invention and disposable incontinence products known to the applicant. The selection of the outer cover stock of the pad so that it can engage the hook fasteners of the reusable pull-on pant is an important part of this simplification of the overall pad construction. While the cloth like outer cover stock is not a loop Velcro-type fastener, it does engage with the hook type fasteners to provide sufficient grip when in the operative position to prevent the pad from dislodging.

The hook fasteners may be located in any suitable locations on the inside surface of the body of the pant given the body shape and size of the person. The selection of the locations of the hook fasteners is discussed further below.

Typically the hook fasteners are not positioned on the inside surface of the body of the pant in the crotch region. This is preferable as any hook type fastener located between the legs is likely to be very irritating due to the physical hardness of the materials needed to construct the hook fasteners. That is, this invention is specifically designed for incontinence pads which are designed to extend to the body of the pant and would not be suitable for feminine sanitary pads which are generally confined to the crotch area.

In addition, the overall external appearance of the fitted product is such that it would not be immediately apparent, if apparent at all, that the person is wearing the product. This would be particularly the case where the disposable pad is a thin pad made from a highly absorbent material. This is a particularly important issue for children and adults who are understandably self-conscious of appearance. It is likely to be less of an issue for babies.

The term “disposable pad” is understood herein to include absorbent pads that can be used by children and adults during periods of incontinence. The term “disposable pad” includes pads that are compostable and flushable. The term “disposable pad” also includes pads that are non-compostable and non-flushable. In general terms, the term “disposable pad” is understood herein to mean pads that are intended for a single use only.

The word “compostable” is understood herein to mean that the disposable pad will disintegrate through biodegradation so as to meet the Australian Standard AS4454-2012 for composting or the equivalent International Standard.

The word “flushable” is understood herein to mean that the disposable pad can be disposed of via a grinder/macerator unit into a sewage system where it becomes part of the biomass and which in turn can be composted and used as a soil conditioner.

The term “hook fasteners” is understood herein to mean hook fasteners in hook and loop fastener systems, for example systems sold under the trade mark Velcro (Registered Trade Mark). The hook fasteners are in the form of small hooks that can engage and retain a material brought into contact with the hooks, such as an outer cover stock of a disposable pad.

The hook fasteners may be in the form of a hook fastener section comprising a single plastic moulding, which is an all-in-one structure, comprising a backing section and a plurality of hooks extending from the backing section.

Typically, the hook fastener section is just cut to size from a large moulded sheet and then attached to the body of the pant by whatever means is appropriate, such as by stitching the sections to the pant.

There may be a plurality of the hook fastener sections attached to the body of the pant.

The hook fasteners may be any other suitable structure.

The hook fasteners may be located on the inside surface of the body of the pant at a front of the pant. The hook fasteners may be located at selected locations across the inside surface of the body of the pant at the front of the pant. The hook fasteners may be located on the inside surface of the body of the pant at selected locations between a crotch and a waist of the pant. For example, the hook fasteners may be located on an upper section and/or a lower section of the front of the pant.

The hook fasteners may be located on the inside surface of the body of the pant at a back of the pant. The hook fasteners may be located at selected locations across the inside surface of the body of the pant at the back of the pant. The hook fasteners may be located on the inside surface of the body of the pant at selected locations between the crotch and the waist of the pant. For example, the hook fasteners may be located on an upper section and/or a lower section of the back of the pant.

The hook fasteners may be located on the inside surface of the body of the pant at the front and the back of the pant.

Positioning the hook fasteners at the front and the back of the inside surface of the body of the pant (and not in the crotch area) is advantageous because it provides additional upward support for the pad by gripping and holding up both the front and back ends of the pad to prevent it sagging down into the crotch area once full of urine. This is an important feature as a fastening means in the crotch area e.g. the regular adhesive strips used to adhere pads to garments, only provides a means to prevent slippage and offers little “support” to maintaining the pad up close to the body, but relies entirely on the gusset of the outer garment to provide upward support. This may be adequate for sanitary towels which are usually not required to contain in excess of 1 kg or more, but is inadequate e.g. where adhesive strips (or any fastening means) in the crotch area are used for locating incontinence pads in a pant. Pads sagging away from the body are a major cause of embarrassing leakage from pads and the gussets of stretch pants are not able to provide sufficient support to prevent this.

The hook fasteners may be any suitable size and shape. The hook fasteners may be secured to the inside surface of the body of the pant by any suitable means. One option is to stitch the hook fasteners to the inside surface of the pant.

The locations of the hook fasteners may be selected to maximise retention of the disposable pad on the person by the pull-on pant.

There may be at least two of the hook fastener sections spaced apart on the inside surface of the body of a front section of the pant. By way of particular example, there may be three of the hook fastener sections spaced apart on the inside surface of the body of the front section of the pant.

There may be at least two hook fastener sections spaced-apart on the inside surface of the body of a rear section of the pant. By way of particular example, there may be three of the hook fastener sections spaced apart on the inside surface of the body of the rear section of the pant.

The pant may include a front section, a rear section, and a sling section that extends between the front and rear sections and in use extends between the legs of the person.

The sling section may be made from a waterproof material.

The sling section may be made from a stretchable material.

The sling section may be made from a stretchable waterproof material.

The pant is typically formed from a stretchable material to help to closely fit the pant onto the body of the person and to contribute to retaining the pad in position by virtue of the stretchiness of the material.

The outer cover stock of the disposable pad may be a woven or non-woven material, typically a soft fluffy woven or non-woven material, or a similar material to provide a comfortable, typically a soft comfortable, covering while also providing a suitable fibre filament finish which can catch on hook fasteners of the pant. The material may be a biodegradable woven or non-woven material or a similar biodegradable material. Suitable materials for the outer cover stock include by way of example only, viscose (i.e.

a fabric made from regenerated cellulose), cotton or PLA (i.e. polylactic acid bioploymer).

The disposable pad may be a multi-layer laminate construction of (a) the outer cover stock described above, (b) a waterproof film for containing solids and liquids, such as by way of example only a film made from a breathable biodegradable starch-based material, and (c) an absorbent fluff pulp layer, such as made from a non-chlorine bleached material, such as made from farmed trees and/or unbleached fluff pulp or bamboo pulp, that is combined with a super absorbing polymer (“SAP”) for absorbency. The term “fluff pulp”, also called “comminution pulp” and “fluffy pulp” is understood herein to mean a type of chemical pulp made from long fibre softwoods, with important properties being bulk and water absorbency.

The disposable pad may further comprise an inner lining and stand up leak guards, such as made from a compostable non-woven material or a similar compostable material. By way of example only, the inner lining may be made from biodegradable viscose, cotton or PLA.

The disposable pad may be compostable.

The disposable pad may be flushable.

The invention is not confined to the use of compostable and flushable pads.

The construction of the disposable pad is such that it can be made from a compostable material or materials, on readily available disposable pad production lines, minimising costs for set up and production.

### BRIEF DESCRIPTION OF THE DRAWINGS

The present invention is described further by way of example with reference to the accompanying drawings, of which:

FIG. 1 is a perspective view of one, but not the only, embodiment of an incontinence product in accordance with the present invention positioned on a person;

FIG. 2 is a front view of a reusable pull-on pant component of the incontinence product shown in FIG. 1;

FIG. 3 is a slightly enlarged view of the circled region of the pull-on pant shown in FIG. 2 viewed from inside the pull-on pant;

FIG. 4 is a rear view of the pull-on pant shown in FIGS. 2 and 3;

FIG. 5 is a series of views (a), (b), and (c) that illustrate a sequence of steps required to be taken by the person shown in the Figures to replace the pad of the incontinence product shown in FIG. 1.

### DESCRIPTION OF EMBODIMENTS

The incontinence product shown in the Figures is suitable for babies, children and adults and comprises:

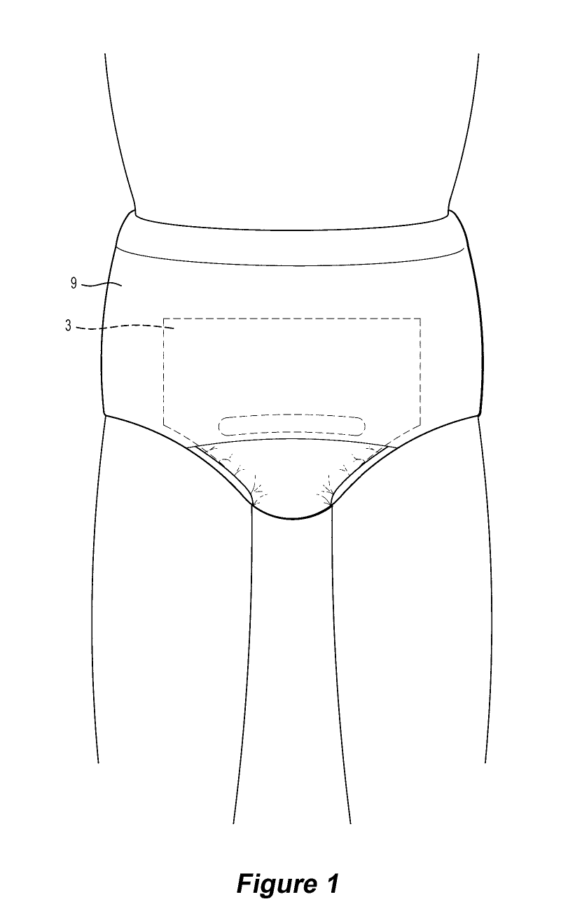

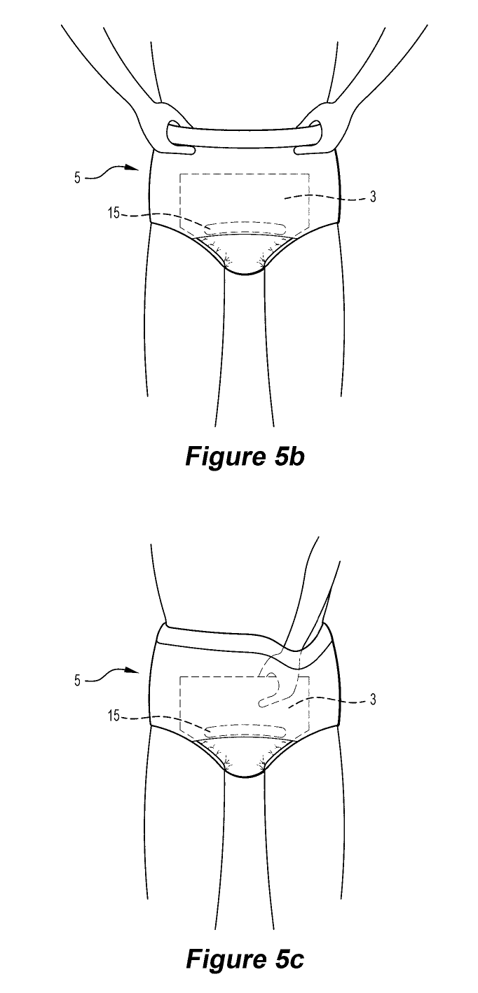

(a) a disposable pad 3 shown in FIG. 5(a) and in outline in FIGS. 1, 5(b), and (c); and

(b) an outer reusable pull-on pant 5.

The outer pull-on pant 5 is a standard pant construction made from any suitable material and includes a waist 9, which is typically elasticised, and a body section 11 that defines leg openings 13. More particularly, the body section 11 includes a front section 21, a rear section 23, and a sling section 25 that extends between the front and rear sections and, in use, extends between the legs of the person. The edges of the sling section 25 are elasticised and formed to snuggly fit around the legs of the person and cup around and support the pad 3 thereby minimise any leakage from the pad. The pant 5 is typically formed from a stretchable material to help to closely fit the pant onto the body of the person and contribute to retaining the pad 3 in position by virtue of the stretchiness of the material.

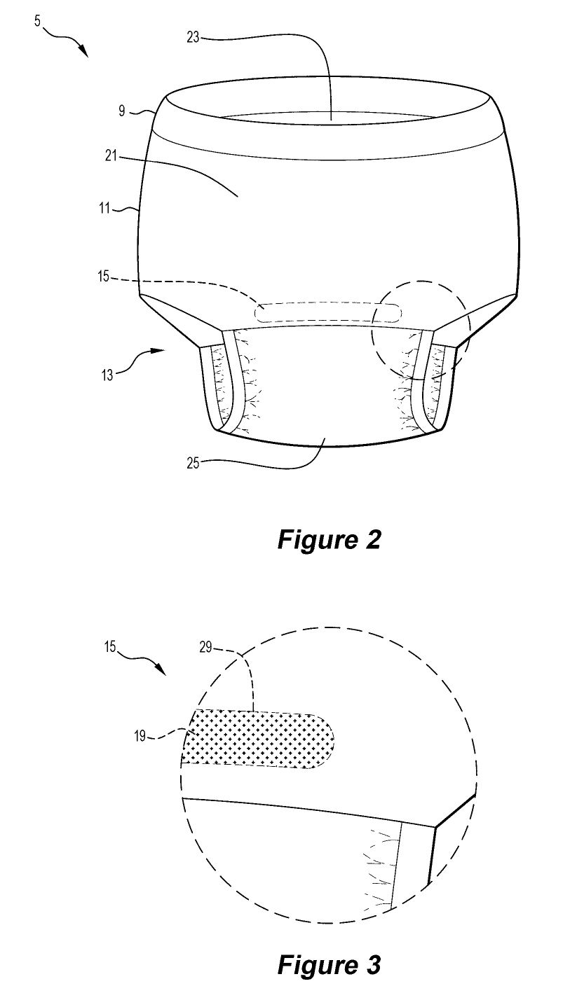

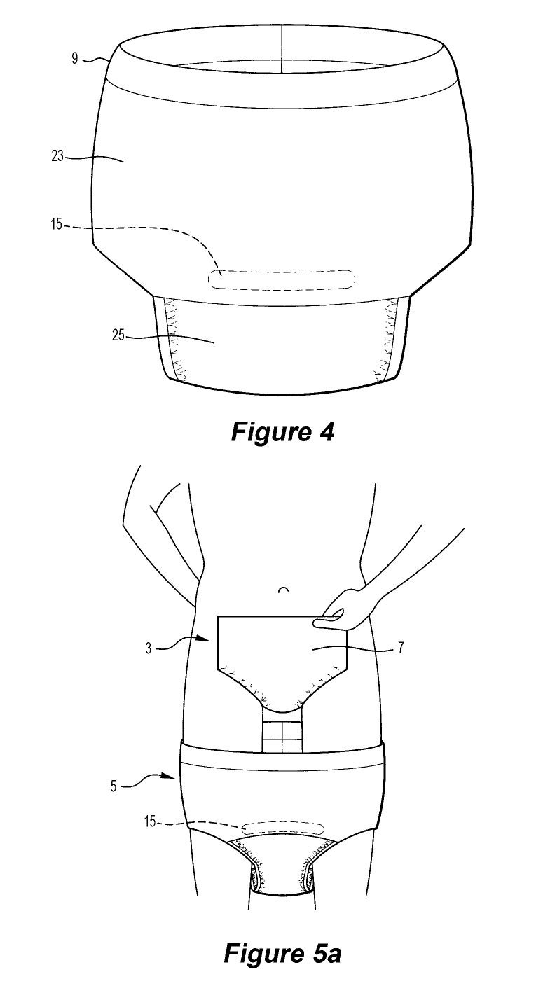

The pant 5 also includes a plurality of hook fastener sections 15 on an inside surface of the front section 21 and the rear section 23 of the body section 11 of the pant 5. Each hook fastener section 15 has a plurality of hook fasteners 19 extending from one surface of a backing section 29 of the section 15. Typically, each hook fastener section 15 is a single plastic moulding which is an all in one structure comprising the backing section 29 and the plurality of fasteners 19 extending from the backing section. The hook fastener sections 15 may be any other suitable construction. The hook fastener sections 15 are secured, for example by being stitched, to the inside surface of the body section 11 of the pant 5 at selected locations at a front and a back of the pant, i.e. at selected locations on the front section 21 and the rear section 23 of the pant. One such section 15 with hook fasteners 19 is shown in FIGS. 2 and 3. This section 15 extends across a lower part of the front section 21 of the pant immediately above the sling section 25 of the pant. It is noted that, typically, there is a section 15 with hook fasteners 19 at a corresponding location on the rear section 23 of the pant. The purpose of the hook fasteners 19 is to engage an outer cover stock (described further below) of the disposable pad 3 and thereby retain the pad 3 in an operative position on the person, as shown in FIG. 1. The size of the hook fastener sections 15 may be selected as required. Whilst the embodiment includes a plurality of hook fastener sections 15, it is noted that in some situations it may be sufficient to provide a single section 15 only.

The disposable pad 3 is shaped to fit around the crotch region of the person wearing the product so as to conform closely to the crotch region to maximise capture of waste and to make it less apparent than would otherwise be the case that the person is wearing the incontinence product. Minimising the visual impact of the product can be a particularly sensitive issue for children and adults.

The pad 3 includes elasticised sides to help to shape the pad 3 to conform to the shape of the crotch region to snuggly hold the pad 3 against the person. The elasticised sides are also formed to define guards that minimise leakage when the pad 3 is held in an operative position.

The pad 3 is a multi-layer laminate construction that is formed from sheets of material.

The pad 3 comprises an outer cover stock 7 (see FIG. 5(a))made from a soft fluffy biodegradable non-woven cloth like material, such as viscose, cotton or PLA, to provide a soft comfortable covering while also providing a suitable fibre filament finish which catches on the hook fasteners 19 of the pull-on pant 5.

The disposable pad 3 further comprises a waterproof film (not shown) for containment of solids and liquids. The film is made from a breathable biodegradable starch-based material.

The disposable pad 3 further comprises a non-chlorine bleached fluff pulp (as described above) layer (not shown) made from farmed trees (or unbleached fluff pulp from farmed trees or bamboo) that has been combined with a super absorbent polymer “SAP” for absorbency. Regular petrochemical based SAP can be replaced with a SAP derived from sustainable resources once a suitable product has been developed. SAP can be beneficial to agriculture where it acts as a “water storing granule”, or if composted it has been shown to biodegrade by the combined actions of white rot fungi and bacteria.

The disposable pad 3 further comprises an optional removable flushable liner (not shown) that can be used to aid in disposing of faecal matter hygienically.

Alternatively, the whole pad 3 can be disposed of hygienically via a macerator/grinder unit which flushes it into the sewer where it goes for treatment and becomes part of the biomass which in turn is beneficially used for application in agriculture, or composted and then used for beneficial use in agriculture as a soil conditioner. The macerating units are readily available and used for disposal of regular non-biodegradable ‘plastic’ disposable pads, however these types of pads are adding large quantities of non-biodegradable plastic particles to our environment which is a problem for agriculture use because of its cumulative effect and potential harm to organisms.

The disposable pad 3 further comprises an inner lining (not shown) which in use contacts the person wearing the disposable pad 3. The inner lining is made from a non-woven material, such as biodegradable viscose, cotton or PLA. This material is an alternative to the traditional polyethylene non-woven material used in current disposable pads.

Polyethylene is regularly used for the inner lining of disposable pads (and nappies) because of its softness and non-absorbing properties, thus providing a stay-dry surface that does not wick liquid from inside the disposable pad 3 to the outside. It also is generally non-irritating when in contact with the skin.

A compostable non-woven material is preferred by the applicant on environmental grounds, although it is emphasised that the present invention also extends to the use of non-compostable materials for the inner lining.

Compostable non-woven materials with similar stay dry and soft properties to polyethylene are made using alternative materials.

FIGS. 5(a) to (c) illustrate the sequence of steps required to be taken by the person shown in FIG. 1 to replace the pad of the incontinence product shown in the Figure with a new pad.

With reference to FIG. 5(a), a first step involves the person pulling down trousers or stockings and the outer pull-on pant 5 and removing and discarding a current pad 3 and positioning a new disposable pad 3 as shown in the Figure. This step does not require the person to remove trousers or stockings to replace a pad 3. Once a new pad is positioned as shown, the person then pulls up the outer pull-on pant 5 as shown in FIG. 5(b) so that the pant 5 is positioned over and retains the new pad 3 in position. As illustrated in FIG. 5(c), the person can then re-position the new pad 3 and the pant 5 relative to each other so that the hook fasteners 19 on the inside of the pant 5 engage the outer cover stock material of the new pad 3 as required to securely retain the new pad in position. The person then pulls up outer clothing, such as trousers and stockings.

It can readily be appreciated that with this combination of pad 3 and pant 5, the pant 5 retains the pad 3 in position, by virtue of engagement of the hook fasteners 19 with the pad 3 and the close-fitting construction of the pant 5.

The above-described disposable incontinence product of the invention has a number of significant advantages.

The fitting sequence is simple and straightforward. It is possible to replace existing pads quickly and easily, with minimal disturbance to clothing. In particular, it is not necessary for the person to remove trousers or stockings to replace a pad 3.

In addition, the overall product design and construction is simple and effective. The pull-on pant 5 is suited particularly well to retaining the disposable pad 5 in position on the person. The use of the hook fasteners 19 on the pull-on pant body 11 rather than on the disposable pad 3 makes the construction of the pad 3 straightforward. This is a significant difference between the product of the invention and disposable incontinence products known to the applicant.

In addition, the positioning of the hook fasteners 19 at the front and back of the body of the pant 5 (and not in the crotch area) also provides additional upward support for the pad 3 by gripping and holding up both the front and back ends of the pad 3 to prevent it sagging down into the crotch area once full of urine. This is an important feature as a fastening means in the crotch area e.g. the regular adhesive strips used to adhere pads to garments, only provides a means to prevent slippage and offers little “support” to maintaining the pad up close to the body, but relies entirely on the gusset of the outer garment to provide upward support. This may be adequate for sanitary towels which are not required to contain in excess of 1 kg or more, but is inadequate e.g. where adhesive strips (or any fastening means) in the crotch area are used for locating incontinence pads in a pant. Pads sagging away from the body are a major cause of embarrassing leakage from pads and the gussets of stretch pants are not able to provide sufficient support to prevent this.

In addition, the overall external appearance of the fitted product is such that it would not be immediately apparent, if apparent at all, that the person is wearing the product. This would be particularly the case where the disposable pad is a thin pad made from a highly absorbent material. This is a particularly important issue for children and adults who are often self-conscious of appearance.

Many modifications may be made to the embodiment of the product of the present invention described above with reference to the accompanying drawings without departing from the spirit and scope of the invention.

For example, whilst the embodiments of the nappy bodies are described in the context of being manufactured from compostable materials, the present invention is not so limited and extends to disposable materials generally.

1. An incontinence product for a baby or a child or an adult comprises:
(a) a disposable pad that is shaped to fit around a crotch region of a person wearing the product; and
(b) a reusable outer pull-on pant having hook fasteners on an inside surface of a body of the pant to engage an outer cover stock of the disposable pad and thereby retain the pad in an operative position.

2. The product defined in claim 1 wherein the locations of the hook fasteners are selected to maximise retention of the disposable pad on the person by the pull-on pant.

3. The product defined in claim 1 or claim 2 wherein the hook fasteners are located on the inside surface of the pant at a front and a back of the pant.

4. The product defined in claim 1 wherein the pant includes a plurality of hook fastener sections secured to an inside surface of the body of the pant.

5. The product defined in claim 1 wherein the pant includes a front section, a rear section, and a sling section that extends between the front and rear sections and in use extends between the legs of the person.

6. The product defined in claim 5 wherein the sling section is made from a waterproof material.

7. The product defined in claim 5 wherein the sling section is made from a stretchable material.

8. The product defined in claim 5 wherein the sling section is made from a stretchable waterproof material.

9. The product defined in claim 1 wherein the outer cover stock of the disposable pad is a woven or non-woven material, typically a soft fluffy biodegradable woven or non-woven material, or a similar material to provide a covering, typically a soft comfortable covering, while also providing a suitable fibre filament finish which can catch on hook fasteners of the pull-on pant.

10. The product defined in claim 1 wherein the disposable pad is a multi-layer laminate construction of (a) the outer cover stock, (b) a waterproof film for containing solids and liquids, such as made from a breathable biodegradable starch-based material, and (c) an absorbent fluff pulp layer, such as made from a non-chlorine bleached material, such as made from farmed trees and/or unbleached fluff pulp or bamboo, that is combined with a super absorbing polymer (“SAP”) for absorbency.

11. The product defined in claim 10 wherein the disposable pad further comprises an inner lining and leg guards, such as made from a compostable non-woven material or a similar material.

12. The product defined in claim 1 wherein the disposable pad is compostable.

13. The product defined in claim 1 wherein the disposable pad is flushable via a macerator/grinder disposal unit.
